(19) United States
(12) Patent Application Publication
(10) Pub. No.: US 2005/0058729 A1
(43) Pub. Date: Mar. 17, 2005

(54) Treatment for bacterial infections and related disorders

(76) Inventor: Jeff J. Staggs, Highlands Ranch, CO (US)

Correspondence Address: JEFF J. STAGGS, 10265 BENTWOOD CT., HIGHLANDS RANCH, CO, 80126, US

(21) Appl. No.: 10/620,794

(22) Filed: Jul. 15, 2003

Related U.S. Application Data
(63) Continuation-in-part of application No. 09/571,644, filed on May 15, 2000, now Pat. No. 6,593,371.
Application No. 09/571,644 is a continuation-in-part of application No. PCT/US93/04763, filed on May 19, 1993.

(51) Int. Cl. A61K 35/78

(52) U.S. Cl. 424/734; 424/760

(57) ABSTRACT

A novel treatment for bacterial infections and related disorders derived from extracts of pepper, ginger, and related plant species containing vanillyl (FIG. 3), and piperidine (FIG. 7) ring structures typical of the pungent principals found in pepper, and ginger. The pepper extracts, which also possess antifungal properties are demonstrated in the topical treatment of bacterial infections affecting the skin eyes, and ears.

### CROSS REFERENCE TO RELATED APPLICATIONS

This is a continuation-in-part of continuation-in-part application PCT/US93/04763 International Filing date 19 May 1993, which is also U.S. PCT National Stage application Ser. No. 08/338,489 filed 18 Mar. 1997 which became U.S. Pat. No. 6,063,381 all of which claim priority from U.S. National application Ser. No. 07/886,640 filed 21 May 1992 now abandoned. This application is with application Ser. No. 09/571,644 which became U.S. Pat. No. 6,593,371 issued 15 Jul. 2003.

### TECHNICAL FIELD

The invention relates to a novel treatment for bacterial infection disorders in humans and animals derived from extracts of pepper and ginger and chemically related plant species.

### BACKGROUND OF THE INVENTION

Humans and animals play host to billions of microorganisms. Among these microorganisms that inhabit our bodies and our environment are a large number of bacteria, most of which are harmless, or even helpful.

A relatively small number of bacteria are potentially pathogenic in nature, capable of causing infections which result in a myriad of diseases that range from mild to life threatening.

Less serious, yet annoying infections of the eyes, skin, sinuses, gastrointestinal tract, and dental plaque are often caused by certain bacteria. Infections of the ears and throat commonly cause considerable pain accompanied by fever resulting in further health complications and more serious illness with younger children being especially hard hit.

Bacteria are often the cause of various urinary tract infections including bladder infections, vaginal infections, and many sexually transmitted diseases.

More serious, and often life threatening diseases involve internal bacterial invasion of the lungs resulting in pneumonia, meninggitis, and sepsis.

Sepsis, an infection of the blood leading to effects an estimated 750,000 patients in the U.S. alone, with an average cost of treatment $22,000 per case $17 billion per year. 32,000 deaths annually make it the tenth leading cause of death in the U.S. However, researches believe the number is closer to resulting in 225,000 deaths, more than breast and colon cancer combined and edging out stroke as the third leading cause of death.

In 2002, the CDC and Emory University released a study that found that the rate of sepsis infection rose an average of 16% per year in the two decades between 1979 and 1999 for a total increase of 329%.

In clinical trials of more than 1,700 of the sickest patients, the best available drug Xigris dropped death rates by only about 6%.

Immune supressed individuals are at greatest risk of infection. These include organ transplant patients, cancer patients, trama patients, burn patients, diabetics, and those with AIDS or pneumonia.

Staph infections from invasive procedures such as IV and catheter insertion sites and surgical incisions from hospitals, nursing homes, prisons, and other institutions often become produce chronic infections that are increasing difficult to avoid or control. Antibiotic therapy too often fails to stem the spread of the infection, often resulting in the need to for surgical removal of diseased tissue to contain the further spread of the infection.

Other serious so called “flesh-eating” strains of bacteria are emerging with increased frequency resulting in serious illness and death in greater numbers.

Tuberculosis, often considered to be a disease of the past is the leading killer of people with AIDS, and the leading cause of death among young adults worldwide with 2 million fatalities annually.

Still other baterial infections result in abcessess, and the list goes on and on.

For the average person, a minor injury such as a burn, or wound is effectively treated with washing, and application of a topical antiseptic. In this regard, antiseptics are effective to reduce the number of potentially infectious microbes at the site of the wound to allow the body's own responses to fight off infection and heal the injury. Any infection tends to be minor and self-limiting, as the wound heals.

If on the other hand infection sets in, and the wound does not heal, topical antiseptics will no longer prove effective. The infection moving into deep layers beneath the skin, and beyond the reach of topical treatments, it will become necessary to administer systemic antibiotic therapy to control the infection. Topical antiseptics are generally only to prevent, and not cure infection, and few effective topical antibiotics that do exist for eye and ear infections are considerably less effective than systemic therapy.

Similar injuries exposed to a hospital environment can have a far more serious impact. Such institutions are breeding grounds for highly virilent drug resistant pathogens.

It is the opinion of the inventor that this is why serious and fatal sepsis cases go unreported. Administrators do not want to admit that the mere institutional environment of hospitals, nursing homes, jails and prisons themselves are responsible for so much serious illness and fatalities.

The few prior art topical antibiotics that do exist are of limited application, and usually less effective than the more costly systemic therapy.

Systemic prior art antibiotic drugs, whether oral or intravenous cause many undesirable side effects. Common side effects include hearing loss, vertigo, gastrointestinal upset, nausea, vomiting, diarrhea, allergic reactions, skin rashes, fever, chills, decrease in white cell count, sensitivity to sunlight, staining of teeth, jaundice, hepatitis crystals in urine, elevated uric acid levels in blood, low blood pressure, eye and nerve damage, and damage to internal organs such as the brain, liver, and kidneys with the possibility of failure.

An even greater problem facing prior art antibiotic drugs is the loss of effectiveness against newly emerging drug resistance pathogenic strains of bacteria of increased virulence.

An eight-state survey by the Harvard School of Public Health found that Penicillin resistance to strep strains rose from 21.7% in 1996 to 26.6% in 1999, and from 10.8% to 20.2% for erythromycin within the same period. The study concluded that as many as 40% of strains of Streptococcus pneumoniae, a common cause of meningitis, sinusitis, ear infections, pneumonia and others will be resistant to both penicillin and erythromycin by the summer of 2004.

A Denver area pediatrician found that as of 2002, 60% of her patients with Streptococcus pneumoniae infections had drug resistant strains

Such is the case with all classes of antibiotics across the board. As new antibiotic drugs tend to be analogs of older drugs, the most effective measures being undertaken to counter drug-resistance is to attempt to avoid over prescribing. At best, such strategy will slow drug resistance by fractional percentages and do little to avert the enevitable. Certainly no good solution to drug resistant bacteria exists among the prior art exists as the crisis looms.

The problem is further compounded by the overuse of antibiotics in livestock feed and it's impact effecting our food supply.

Added to the misery of illness and death is the adverse economic impact that bacterial diseases exact.

The high cost of treatment adds further to the already exorbitant cost of healthcare borne by employers, government, and ultimately the patient through added medical bills, increased insurance premiums and higher taxes. Repeated physician visits reduce worker productivity, and leisure time.

At particular disadvantage are low-income individuals who cannot afford treatment. As 1 in 6 Americans cannot afford health care insurance, this group is most likely to suffer more serious illness as a result of a lack of earlier treatment with disasterous consequences. The situation is yet far worse for the poor of developing countries where the cost of antibiotic therapy is out of reach often exceeding average family income.

Today, more than every before, the importance of having medications that are not only safe, and effective, but cost effective as well is critical not only to our health, but our very way of life.

### BRIEF SUMMARY OF THE INVENTION

One object of the invention is to provide a highly effective topical antibiotic treatment able to quickly resolve local bacterial infections such as those that result from surgical incisions without the need for systemic antibiotic drug therapy.

The advantages of such a treatment are more immediate relief of illness for the patient. A more quickly resolved local infection reduces or eliminates the risk that the infection will spread and generate into a more serious invasive infection. The harmful and unpleasant side effects and inconvenience associated with longer-term prior art systemic antibiotic therapy is eliminated, along with the associated health risks and complications. The present invention could be used as a first line treatment in conjunction with prior art systemic therapy implemented as a precautionary measure to guard against subsequent invasive infection, or simply for possible synergetic effects. In either case, subsequent courses of antibiotics would not be necessary, as they often are with prior art treatments alone.

In either case, a reduction in the use of prior art systemic antibiotics supports efforts by physicians, government agencies and public health care providers to reduce unnecessary use of antibiotics which contribute to the emergence of drug-resistant strains of pathogenic bacteria.

Considerable savings in the cost of health care is another important advantage of the present invention. The ability to resolve local infections with a few topical treatments alone is considerably less costly than coarse after coarse of oral, or intravenous antibiotics as is now required by prior art treatments with often disappointing results. Full implementation of the invention would translate into savings for all health care providers and lower insurance premiums for patients.

A treatment such as the present invention that saves the misery of millions of sufferers, is low in toxicity, low in cost, safe, more convenient to use, and is affordable to the poor offers much broader commercial feasibility over prior art treatments.

A treatment that saves billions of dollars annually in patient productivity, healthcare costs, and capable of becoming a model for demonstrated savings in commercial healthcare, and government sponsored programs such as Medicare, and Medicaid. An economically feasible veterinary treatment for livestock, pets, companion, and other animals of broadened commercial appeal.

Also provided is an important research tool applicable toward developing improved treatments for other more serious and life-threatening systemic infections along with a new class of antibiotics of increased effectiveness against prior art drug-resistant pathogens.

Pepper extracts appear to possess multiple therapeutic actions in addition to direct antibacterial/antimicrobial action. Case observations suggest general healing (vulnerary), keratolytic, immunostimulation and modulation, adjuvant, drug delivery, antitoxin, antihistimine, antiitch, and prophylactic properties beyond direct antimicrobial. Though not tested to date for antibacterial activity in vitro, in vitro antifungal screens prove proportionally increased potency against lethal drug-resistant fungi strains in addition to unparralled effectiveness in treatment of actual disease in afflicted patients. Reference is made to U.S. Pat. No. 6,593,371 with regard on treatments for wart related disorders, and U.S. Pat. No. 6,063,381 with regard to antifungal uses, and related therapeutic properties and actions that are believed to be equally applicable in treating bacterial infections. Antifungal medications in general, whether prior art or future art may also possess properties useful in treating bacterial infections as disclosed herein. More specifically: nystatin, amphotericin B, griseofulvin, tolnaftate, and all the “azole” derivative antifungals including, but not limited to clotimazole, miconazole, econazole, ketoconazole, along with the “triazole” class including, but not limited to such derivatives as fluconazole, terconazole, and itraconazole ect.

It appears that the high nutrient concentration found in pepper including vitamins, minerals, carotenoids, lipids, various stress metabolite including phytoalexins and other compounds not specifically identified here assist the above therapeutic effects in addition to the pungent compounds. Pepper compounds are safe, and have been in widespread use as food for thousands of years and do not induce illness, harmful side effects, or injury as do prior art treatments.

As a generally recognized as safe (GRAS) listed nutrient food compound, pepper medications are ideal for livestock, pet, and companion animal use in addition to human. Sytemic, as well as topical medications to control a variety of other bacterial infections can be developed including feed additives to prevent disease.

The veterinary market for treatment of bacterial diseases can be greatly broadened given the high effectiveness, low toxicity, and very low cost of my medication. Many of these disorders need no longer afflict livestock, pets, companion or other animals as before.

The current invention enlarges the scope of product possibilities by making treatment of these bacteria related disorders easier, and more economically feasible.

The many therapeutic properties, and beneficial components found in pepper provide ideal prospects for developing systemic treatments for the more serious, and often life threatening disorders such as sepsis. The high antibiotic effectiveness and antitoxin actions of pepper compounds without the toxic drug side effects typical of prior art drugs that further endanger the health of the patient provide unequalled advantage. With the steadily rising number of cancer, AIDS, and immunosuppressant drug treatment cases reported now, and anticipated for the future, pepper compounds provide an important research tool in treating those most vulnerable to these more serious, life threatening conditions.

Commercial implementation of this topical antibacterial treatment alone can have major impact by making affordable a certain cure to even the poorest people of both industrialized, and developing countries now excluded from care because of the high cost, and increasing low effectiveness of prior art treatments. A complete cure of infections that do not require an attending physician, and for less than one penny on the dollar of the cost of prior art treatments. This will bring relief to many millions of sufferers, rich and poor alike while expanding the consumer demand base for the product accordingly.

Full-scale implementation of medications that resolve lesser bacterial infections, and prevent more serious subsequent infections would save billions of dollars in Gross National Product in the U.S. alone, not to mention the world.

This enhanced level of safety, effectiveness, and dramatic cost savings of this medication should serve as a model to both private managed care programs including government healthcare programs such as Medicaid, and Medicare wherein billions of dollars in medical expenditures are saved while providing the best care for recipients.

In conclusion, this novel antibiotic treatment can save our nation and many nations of the world millions of dollars each day in medical costs, and lost productivity. It provides highly lucrative new products for commercial exploitation in the areas of human and animal health. It provides an important research tool in the prevention, and treatment of serious and life-threatening disease that will save the lives of man and animal alike. It makes now available speedy relief to hundreds of millions of suffers.

### BRIEF DESCRIPTION OF THE DRAWINGS

FIG. 1 is a molecular diagram of phenol.

FIG. 2-13 show molecular diagrams of compounds of the current invention.

FIG. 2 is a molecular diagram of ortho-methoxyphenol.

FIG. 3 is a molecular diagram of vanillyl.

FIG. 4 is a molecular diagram of 3-methoxy-4-hydroxybenzylamine.

FIG. 5 is a molecular diagram of vanillylamide.

FIG. 6 is a molecular diagram of the capsaicinoids.

FIG. 7 is a molecular diagram of piperidine.

FIG. 8 is a molecular diagram of the pungent alkaloid principals of pepper.

FIG. 9 is a molecular diagram of eugenol.

FIG. 10 is a molecular diagram of curcumin.

FIG. 11 is a molecular diagram of gingerol.

FIG. 12 is a molecular diagram of resiniferatoxin.

FIG. 13 is a molecular diagram of tinyatoxin.

### DETAILED DESCRIPTION OF THE INVENTION

A medicinal preparation of pepper, and its active constituents may be administered in a wide range of concentrations, and conventional drug vehicles and carriers in the treatment of superficial, cutaneous, subcutaneous, systemic, and internal bacterial infections afflicting humans, animals, livestock, cattle, pets and companion animals in general. Pepper compounds are essentially broad-spectrum in nature, but have optimal narrow-spectrum application as would be expected according to more specific applications as listed below in the treatment of all types of infections, or prevention of infection.

More specifically, but not limited to infections that result from any invasive medical procedures such as the site of surgical incisions, catheters, IV's, and hypodermics, blood samples and biopsises.

More specifically, but not limited to general cellulitis, ear infections, eye infections, sinusitis, food poisoning, skin infections, furuncles, folliculitis, scalded skin syndrome, general wound infections, necrotizing fascitiitis (“flesh eating disease”), lung infections, pneumonia, toxic shock syndrome, actinomycosis, nocardiosis, meningitis, and sepsis.

More specifically, but not limited to infections caused by gram-positive and gram-negative bacteria including Staphylococcus, Staphylococcus aureus, Hemophilus, Hemophilus influenzae, Pseudomonas, Pseudomonas aeruginosa, Streptococcus, Streptococcus pneumoniae, Streptococcus Group A, Group B, Group C, Group D, Group G, Mycobacterium, Mycobacterium tuberculosis, Clostridium, and Enterobacteriaceae.

More specifically, but not limited to infections that are caused by antibiotic drug resistant strains of bacteria.

For treating all types of patients more specifically, but not limited to trama patients, burn patient, cancer patients, patients taking organ transplant drugs, diabetics, immune suppressed patients, and patients with AIDS.

The preparations described below are made from an ordinary commercial grade of ground cayenne pepper (Capsicum frutescens), or black pepper (Piper nigrum) to serve as an indicator of approximate concentration within each carrier. Their equivalents may also be prepared from pepper oleoresin, which is available commercially in a wide range of concentrations, and pungency. Component parts such as the pungent principals are inclusive of pure natural, synthetics, and artificial form.

The term “pepper”, or “pepper compounds” are used somewhat generically, and is inclusive of related botanicals having similar constituents. For example, the Zingiberaceae family including ginger (Zingiber officinale), turmeric (Curcuma longa), cardamon (Elettaria cardamomum), Melegueta pepper (Aframonum melegueta), members of the Euphorbia genus including Euphorbia resinifera, poinsettia (Euphorbia pulcherrima), clove (Eugenia aromatica), allspice (Pimenta oficinalis) and others such as vanilla. These also may be prepared in the same way as pepper by following the general procedures outlined in the illustration below. Higher concentrations may also be used, as many of these lack the pungency of actual pepper, and sensory irritation is less of a concern.

Included among this list of botanicals are the other members of the Solanaceae pepper family including members of the Capsicum genus with species annuum, baccatum, and longum. These include hot pepper and sweet pepper varieties including paprika.

Among the Piperaceae family; species of the Peperoma, and Piper genus which include species retrofractum, nigrum, longum and methysticum. Other species of plants having similar chemistry may also be used in place of the above.

Variations in performance of each preparation will vary basis botanical source, solvent used for extraction, concentration of extract, and carrier type in relation to the particular pathogen, host, and site of infection. The scientific literature may be consulted for more detailed investigations as to chemical properties, solubility, separation, and quantitation of constituent compounds.

As the medicinal preparations listed below are for the most part crude botanical drugs, it should be noted that the present invention is inclusive of any and all therapeutic agents found in the botanical source. While the pungent agents themselves have antibacterial properties, these are not the sole antibacterial agents. The totality of therapeutic actions in treating illness is quite complex, and it would not be possible or practical to seek to identify each one in detail.

The discussion below with regard to the chemistry of the pungent compounds is intended both to identify possible antibacterial components for isolation, recombination, re-proportion, new combinations, to serve as guides in developing new chemical entities, and to serve as a marker to aid in identification of other botanical sources not specifically listed here.

With reference to red peppers for example, see procedures described in the article “Separation and Quantitation of Red Pepper Major Heat Principals by Reverse Phase High-Pressure Liquid Chromatography” by Patrick Hoffman et. al., in the Journal of Agricultural Food Chemistry 1983, Vol. 31, pages 1326-1330.

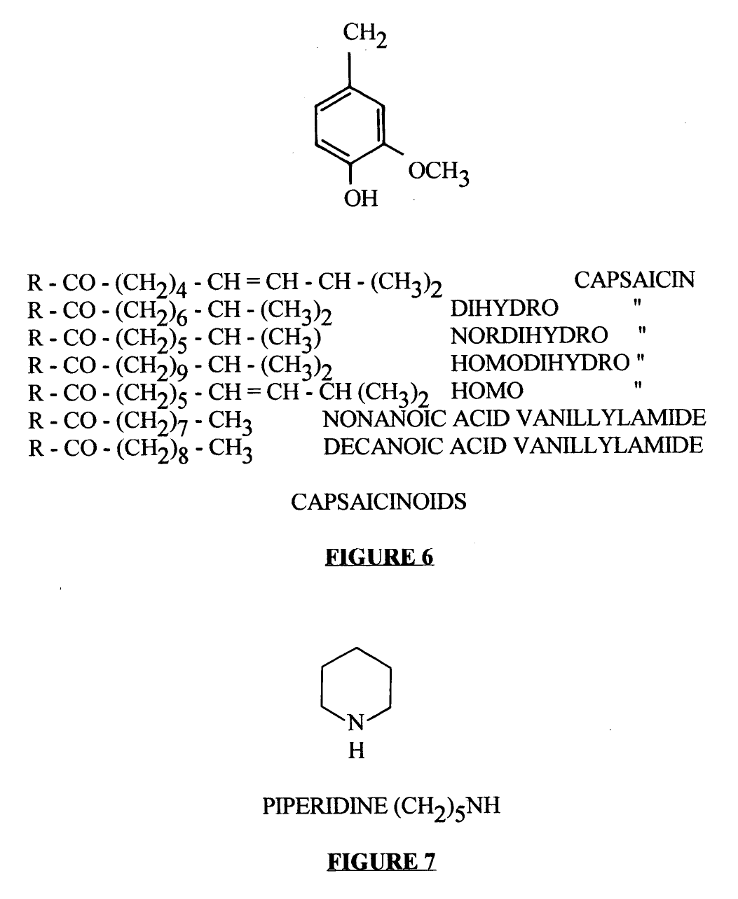

The major capsaicinoids (FIG. 6) include:

Capsaicin. C₁₈H₂₇NO₃
N-[(4-hydroxy-3-methoxyphenyl)methyl] 8-methy-6-nonenamide). Dihydrocapsaicin. C₁₈H₂₉NO₃
(N-[(4-hydroxy-3-methoxyphenyl)methyl]-8methylnonanamide).
Norcapsaicin. C₁₇H₂₅NO₃
(N-[(4-hydroxy-3-methoxyphenyl)methyl]7-methyl-5octenamide).
Nordihydrocapsaicin. C₁₇H₂₇NO₃
(N-[(4-hydroxy-3-methoxyphenyl)methyl]-7-methyloctanamide.
Homocapsaicin. C₁₉H₂₉NO₃
(N-[(4-hydroxy-3-methoxyphenyl)methyl]-9-methyl-7decenamide).
Homodihydrocapsaicin. C₁₉H₃₁NO₃
N-[(4-hydroxy-3-methoxyphenyl)methyl]-9methyldecanamide). N-vanillyl-n-nonamide. C₁₇H₂₇N₃
(N-[(4-hydroxy-3-methoxyphenyl)methyl]-n-nonamide). Nonanoic acid vanillylamide. C₁₇H₂₉NO₃
Decanoic acid vanillylamide. C₁₈H₃₁NO₃

Other capsaicinoids, identified in research literature as trace elements may be used in medicinal preparations along with other analogous including synthetic compound.

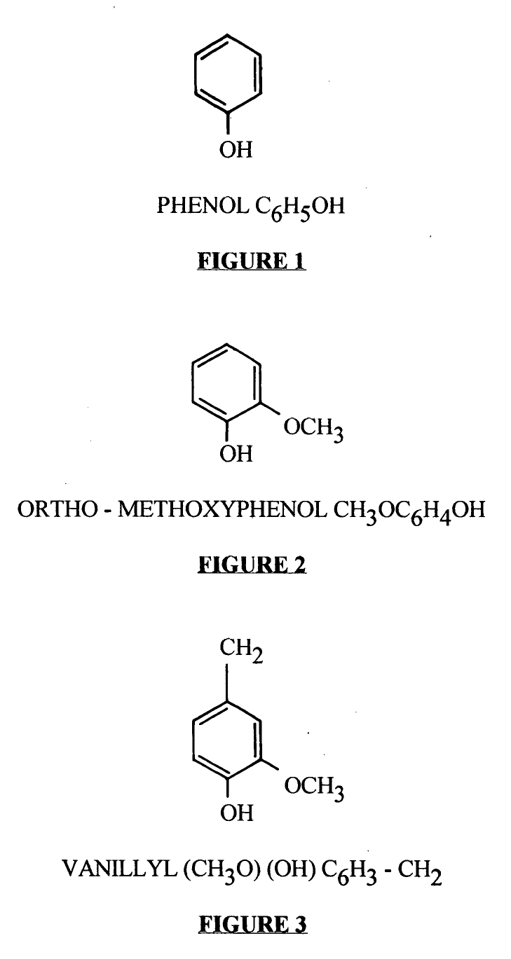

Capsaicinoids are acid amide derivatives of Phenol (FIG. 1). The characteristic pungent, irritating sensory effects of these compounds are typical of acid amides, whether derived from phenol, or piperidine (FIG. 7)

Phenol (FIG. 1), though lacking pungent flavor, is highly corrosive, caustic, and toxic, deriving many of its properties from its basic benzene structure. While this gives phenol certain antimicrobial properties, it is generally considered unsuitable for therapeutic use in man, and animals, due to it's and irritating effects on tissue.

With the addition of a methoxy group (OCH3) to phenol, methoxyphenol is formed. In the ortho position, we have ortho-methoxyphenol (FIG. 2), also known as guaiacum, an extract obtainable from trees of the Guaiacum genus. The effect of this methoxy group in part is an increase in aromacy, and a decrease in toxicity, and caustic properties otherwise existing in phenol, yet without apparent decrease in antimicrobial properties. The attachment of hydrocarbon groups to the ring structure, to form higher analogues apparently increases the antimicrobial properties of methoxyphenol, and phenol. It is presumed that the meta, or para isomers of methoxyphenol have similar properties to the ortho, in like manner to the similarities between the phenol isomers.

The addition of the methylene group (CH2) in the para position to ortho-methoxyphenol produces vanillyl (FIG. 3). Like phenol, and methoxyphenol, it is presumed that changing the position of the methylene group to form other vanillyl isomers will produce compounds of similar, although not exact properties to that of vanillyl.

The vanillyl structure on which the capsaicinoids are constructed is also typical of the pungent principals found in ginger (Zingiberaceae) species of plants.

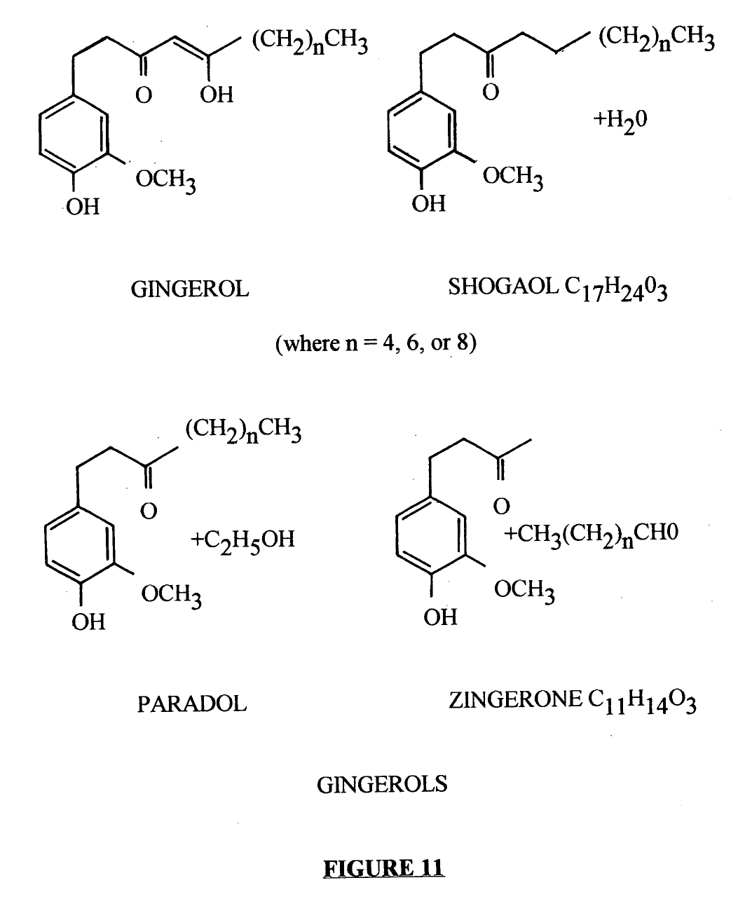

Collectively known as gingerol (FIG. 11): shogaol, paradol, zingerone, gingerol and other analogs, have a different side chain than the capsaicinoids, and lacking an ammonia (NHₙ) group, are neither amines, or amides like the capsaicinoids or piperidine series. Hydrolysis of gingerols yield vanillyl, and a fatty acid side chain, both of which demonstrate like therapeutic properties to the capsaicinoid hydrolytes.

Also members of the ginger, or Zingiberaceae family, turmeric (Curcuma longa L.) contains the compound curcumin (FIG. 10), actually a vanillal derivative differing from vanillyl by one hydrogen (H) atom having an (CH) substituent, rather than a methylene (CH2) in the para position. This analog differs further with a side chain unique from the others. Cardamon, allspice, clove, black pepper, and many others contain eugenol, another vanillyl analog with yet another hydrocarbon side chain.

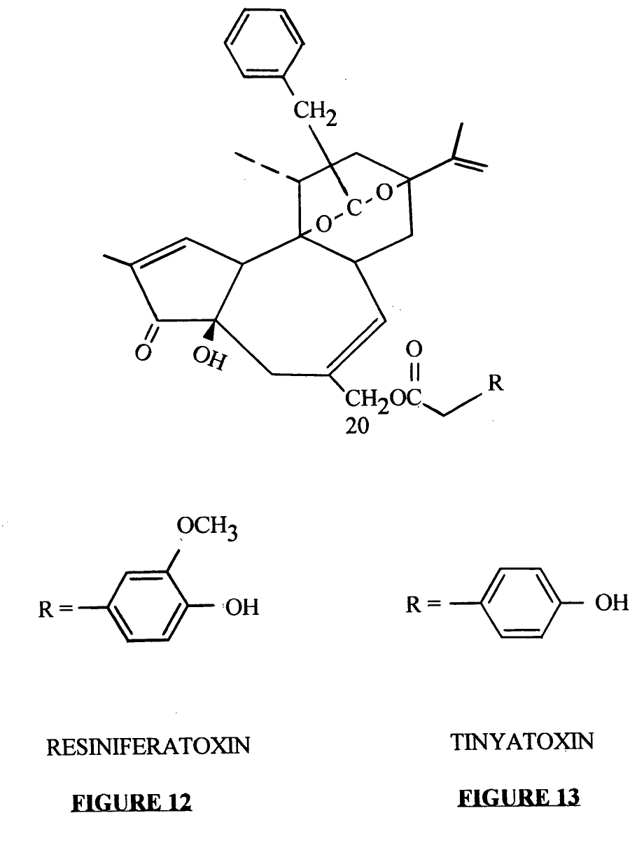

Other botanical sources of vanillyl analogs include gum euphorbium, and extract of certain species of the Euphorbia genus, which contain the capsaicin analog resiniferatoxin (FIG. 12), along with its analog tinyatoxin (FIG. 13) and others.

Replacement of one of the hydrogen (atoms of ammonia (NH3), with vanillyl, and replacement of the other hydrogen (H) atom with an organic hydrocarbon group produces vanillylamide (FIG. 5). In the case of the capsaicinoids (FIG. 6), or capsaicin analogs for example, this organic hydrocarbon group is a chain acid (R′), varying from about 8, to 14 carbon atoms, depending upon the particular capsaicinoid. These side chains, both saturated, and unsaturated add to the pungency of capsicums, and themselves possess antimicrobial properties of their own, without apparently contributing corrosiveness, or toxicity to vanillylamide.

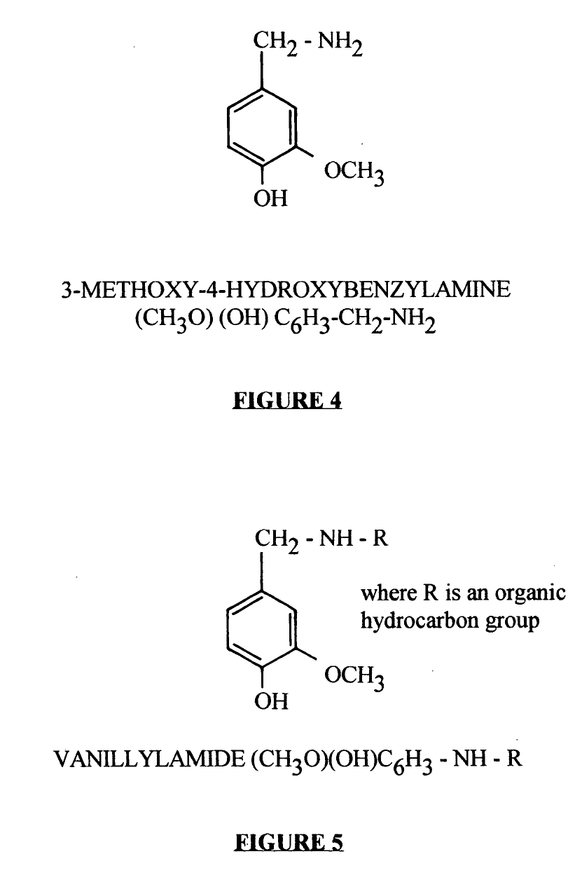

Hydrolysis of capsaicinoids yield active agents as well. The splitting off of the side acid chain, and it's replacement with a hydrogen (atom yields the primary amine vanillylamine, or 3-methoxy-4-hydroxybenzylamine (FIG. 4) from vanillylamide (FIG. 5), in the case of all capsaicinoids. Conversely, the side acid chain, receiving a hydroxy (OH) group, is converted to a fatty acid, and yields a different hydrolyte for each individual capsaicinoid. In the case of capsaicin (FIG. 6), hydrolysis of the side acid chain R′ (FIG. 6) CO—(CH₂)₄—CH═CH—(CH 3)₂ yields iso-decylenic acid COOH—(CH₂)₄—CH═CH—CH—(CH₃)₂.

The piperidine series (FIGS. 7 & 8)), represent a group of analogous alkaloid compounds from which most of the pungent principals found within plants of the Piperaceae family, of which black pepper (Piper nigrum) is a member, are found. Also classified as acid amides, the piperidine series, like the capsaicinoids found in capsicum species, are primarily responsible for the characteristic sharp, pungent taste of black pepper.

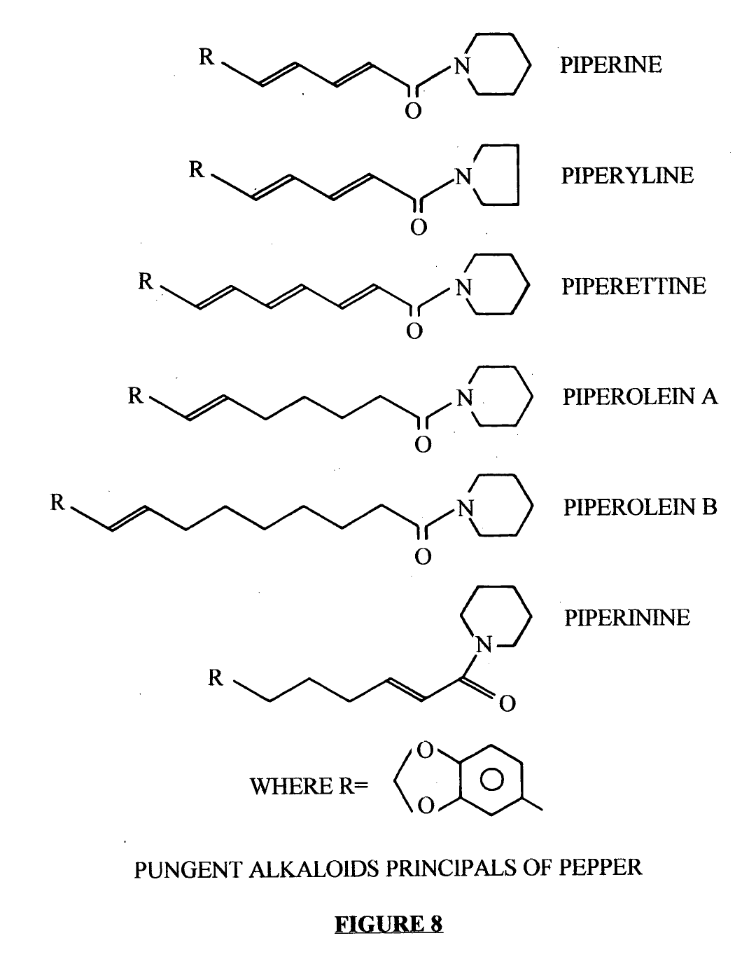

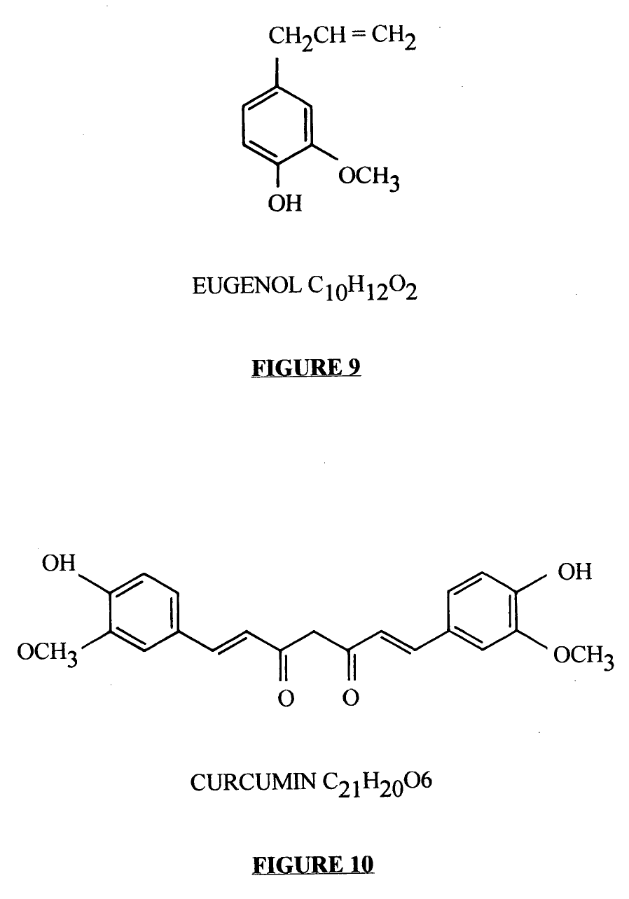

The piperidine ring (FIG. 7) structure is diverse from that of phenol (FIG. 1). Though also a six membered, carbocyclic compound, the piperidine series instead contain one nitrogen (N) hetero atom within the ring. Piperidine is heteroparaffinic, and contains no double bonds. The hetero nitrogen atom within the ring is a contributor to the pungency of these compounds. The attachment of a hydrogen (H) atom to the hetero nitrogen atom within the ring forms the amine structure. Attachment of a hydrocarbon group, in the form of a side acid chain (R″ FIG. 8) attached to a benzene structure establishes the acid amide structure. These compounds include; piperine C₁₇H₁₉NO₃ (FIG. 8), chavicine C₁₇H₁₉NO₃, piperettine C19₂₁03N, piperidine (CH₂)₅H, piperyline, piperolein A, piperolein B. piperanine, and others.

Hydrolysis of the piperidine series, like the capsaicinoids, yield active, pungent compounds. Chavicine, for example is hydrolysed to piperidine, which receives an additional hydrogen (H) atom to form a primary amine, and chavicic acid, which receives the hydroxy (OH) group to form the fatty acid.

Hydrolysis of these capsaicinoid, and piperidine acid amides, as well as the other listed compounds may be accomplished with chemical catalysts, or by boiling a liquid preparation in water. Hydrolysis does not appear to diminish pungency, and in some applications appears to enhance both pungency, and therapeutic action.

The carbonyl group (C═O) side chain substituent, common to all the above compounds (except eugenol) is also believed to be a contributor to therapeutic action.

Other active agents found within capsicum include citric acid, vitamins A, B1, B2, C, and E, iron, potassium and niacin in significant quantities, along with other lipids, and carotenoids including capsanthin, capsorubin, and others. Vitamin C concentrations of 100 milligrams per ounce, are the highest of any natural food compound. Vitamin A content is also high, with 6170 I.U. per ounce.

Still other active agents include any of a number of stress metabolites including phytoalexins and related compounds.

An infusion of pepper is prepared by soaking approximately 4 cm3 (¼ teaspoon) of commercially available ground red, or black pepper, to one liter (1 quart) of water of sensibly comfortable temperature. Set at least ten minutes before use for best results. Strain plant residue before use if desired.

A more potent tea uses about 16 cm3 (1 teaspoon) of ground pepper for each liter (quart) of sensibly comfortable water. Tea may also be prepared from boiling water, or itself be boiled in water before use. Boiling pepper in water assures complete hydrolysis of the pungent principals, which are also active agents. A stronger tea would use the same amount of spice with less water, perhaps ¼ the amount above.

A tincture is prepared by soaking ground red, or black pepper in a solution containing approximately 60% ethanol, and 40% water. Pure ethanol, acetone, chloroform, vinegar (acetic acid), and others may also be used. The fluid volume of the solution may be about three, or four times that of the dry volume of the ground pepper. The mixture should be agitated, at least occasionally, over a period of at least two hours, with maximum extraction being obtained after about six hours. Allowing the mixture to sit over night produces excellent results. Strain off the residual ground pepper.

A preparation of pepper drops is obtained by reducing tincture through heat, or passive evaporation. Drops made by this method are similar in purity to some grades of commercially available oleoresin. Drops appear to concentrate by a factor of about 10 fold.

A pill or capsule is prepared from ground spice, oleoresin, or any of various extracts for oral administration.

A plaster, or poultice is prepared by mixing ground pepper with water, until it has a paste-like consistency that will assure good adherence to the skin, or cloth to which it is applied.

A lotion, a cream, or a shampoo may be obtained by adding to any commercially available shampoo, cream, or lotion, a portion of drops, or tincture equal to approximately 25% of the volume of lotion, cream, or shampoo carrier.

An injection is prepared from a purified version of infusion, tea, drops, or any isolate etc., administered into tissue, blood stream, or spinal fluid hypodermic, or in a fluid stream into body cavities such as rectum, mouth, and throat directly on infected areas.

A powder is pepper in ground form, or extracts mixed.

A pepper impregnated fabric is clothing, including socks, shoe liners undergarments, and athletic wear, or anything that has contact with infected areas of the skin including bandages made from capsicum wool, or any other pepper compound.

Treatment recommendations given below are general guidelines and may be altered to suit specific conditions. If one recommended concentration appears unsuitable, the next graduation should be used.

Consideration as to area of infection, patient sensitivity to the medication and certainly how anxious the patient is to be rid of the infection. Higher concentrations seem most effective.

In the lower concentrations, an infusion may be used where skin sensitivity is high. Infusion works well as a scalp rinse, mouth rinse, a bath for the feet and skin, genital area, or for injection.

In higher concentrations, administrations equivalent to a tincture, a powder, a poultice, and a preparation of drops are recommended where skin sensitivity will permit.

Still higher concentration drops for areas of low skin sensitivity. Drops appear also to have a prophylactic action of greatest duration lasting days after application. Drops made from cayenne pepper are extremely irritating to eyes, mucosa, and more sensitive skin areas, so care should be taken. Drops made from less pungent botanicals should be considered. Drops made from paprika for example, are much better tolerated, and can even be administered full strength to these sensitive areas without undue discomfort. Drops may be placed in capsules for oral administration.

A tea represents a moderate concentration of pepper compounds. It may be used in the same manner as infusion. Tea may also be used in place of the higher concentration carriers, such as drops or tincture.

An injection directly into drained abscesses, intravenous, or in spinal fluid.

A lotion or shampoo may be prepared with any commonly available lotion, or shampoo, and applied to infected areas in its intended manner. Other therapeutic agents, in addition to pepper extracts, may be added to shampoo and lotion. If irritation is a concern, a topical anesthetic, such as lidocaine, or benzocaine may be added to lotion to reduce severity. If skin is very dry, emollients may also be added to lotion.

A pepper aerosol may be used in the treatment of the mouth, throat, or lungs infections. In this administration, care should be taken, as some embodiments can be extremely irritating to the nose, throat, lungs, and eyes, especially when airborne. This is especially true of capsicum aerosol. Less irritating embodiments might be a preferred first choice.

Pepper powder is also very irritating when airborne, and like aerosol, has a more limited medical application than the other carriers.

Smoking powder or oleoresin for lung infections.

The unprecedented effectiveness of the current invention is demonstrated in the treatment of several patients, all infected with chronic bacterial infections.

In the first illustration, a woman in her 40's afflicted with what appears as a possible staph infection over a surgical incision from a cessarian section is routinely healed after a single treatment with a pepper extract.

The woman recalls that while recovering from surgery, a nurse removed the surgical staples from the woman's left toward her right side. The woman recalls this procedure as relatively uneventful, with the exception that the nurse seemed to have more difficulty in removing the last few staples on the right end of the incision, and that their removal was painful. The woman recalls that the same right end of the incision was sore for a number of days, and took longest to heal.

The woman reported that several months after surgery, a round, quarter-sized red area appeared around the right end of the area of the incision. The red area would begin to itch. Within days, the red area would expand toward the left over the entire area of the incision, forming a band approximately three inches wide. In doing so, this band would become more intensely red and itchy, and would generate painful cuts along and on each side of the incision, and oozed foul-smelling pus.

A culture was taken, and a diagnosis of drug-resistant strain of Staph aureus infection was made.

A single application of a lotion made from a tincture of freshly ground black pepper was applied to the infected area. The tincture was made from a 70% isopropal alcohol extraction followed by acetone extraction and evaporated down within a lotion base.

The woman reported that the itching disappeared almost immediately upon application of the lotion with a gradual reduction of soreness to the point of being unnoticeable within less than one hour. The appearance of pus and redness also began to gradually fade away until normal, healthy skin and color was completely restored within a day or two.

This same pattern was observed over a period of five or more years. The infection would reemerge one to three times per year, and resolve in the same manner after a single application of the black pepper lotion.

On a few occasions, the infection was treated a single time with oleoresin of paprika, or a lotion made from red pepper with about the same result. The only observed disadvantage of these two alternative treatment was that the red pepper lotion caused greater burning discomfort that lasted ten or fifteen minutes. The paprika oleoresin had a tendency to stain underclothing, about half the time a second treatment seemed necessary to achieve the same results as with the black pepper lotion.

A diabetic male in his early forties was suffering from a chronic ulcer on the back-side of his left thigh that had further degenerated into a stubborn staph infection (diagnosed).

The man had been on various courses of oral antibiotics over a period of months to control the infection with little success. The infection was spreading, and moving down his leg in red streaks. The doctor decided to operate to remove the diseased area of skin and subcutaneous layers in order to stop the further spread of the infection.

Two weeks after surgery, the surgical incision and surrounding area retained signs of infection, and did not appear to be healing.

The man was given a black pepper lotion as described above and applied the same over the incision and infected areas with astounding results. The man's comment was “ . . . I don't know what you put in that lotion, but I couldn't believe how quickly it healed my infection. Other than the fact that it burned like hell at first, the infection showed improvement immediately. I've never seen anything work anywhere near this well with all the treatments I've gotten from my doctors. I wish I'd had a chance to try your lotion before getting the surgery.”

Several months have passed subsequent to resolution of the infection without reoccurance.

A man in his mid-thirties was diagnosed with a chronic case of cellulitis afflicting the lower part of his left leg around the sock area. A culture revealed a strep infection.

The man was given a black pepper lotion as described above and applied the same over the incision and infected areas with astounding results. The man reported that all symptoms had begun to resolve within the first day of treatment, and that he had not seen anything this effective from his prescription medications.

A fourteen year old girl with a chronic eye infection lasting several months resulting in a pimple-like granuloma on the edge of her lower right eyelid.

The girl's doctor indicates a bacterial infection, probably staph as a probable cause.

A single treatment of black pepper lotion as described above was administered beneath her eyelid and on the granuloma. Within hours the granuloma shrank, and gradually disappeared within about a week and a half of treatment.

A woman in her thirties with a recurrant ear infection is cured with a single treatment of oleoresin of paprika. The woman states that she develops earaches in her right ear that last for many days after and hour or more of exposure to wind. On numerous occassions these earaches have been resolved almost immediately after a single treatment of the paprika oleoresin, and do not reoccur for many months until again exposed to wind.

A woman in her forties with a recurrant sinus infection is cured with a single treatment of oleoresin of paprika. The woman states that such infections tend to develop every few years, with nasal congestion and pain in the upper teeth and sinuses. The woman complained that the paprika oleoresin was a bit too painful the first few minutes after treatment, but nonetheless all symptoms of the sinus infection began to subside almost immediately thereafter, and congestion disappeared completely that same day.

Various black pepper based lotions have relieved numerous infections from minor cuts and burns, and also appears to have pain-relieving properties along with causing much more rapid healing.

Water extracts like teas and infusions show excellent results in relieving various eye infections including nonspecific conjuntivitis, and suspected pinkeye and other infections. Chemical solvent extracts may also be used, but care should be taken to reduce or remove potentially harmful solvents before use.

Oral administration of pepper extracts in the form of capsules or teas have been found to be effective in warding off suspected food poison if taken at the first signs of nausea or other gastrointestinal upset.

1. A method of treating infectious diseases caused by drug-resistant strains of bacteria in a human or an animal host comprising:
Administration to the area of disease a suitable carrier containing an antibacterial agent obtainable from pepper, or an equivalent in a therapeutically effective concentration.

2. A method of treating bacterial infections in a human or an animal host comprising:
Administration to the area of disease a suitable carrier containing an antibacterial phytoalexin obtainable from pepper, or an equivalent in a therapeutically effective concentration.
